(19) United States
(12) Patent Application Publication
(10) Pub. No.: US 2010/0280040 A1
(43) Pub. Date: Nov. 4, 2010

(54) SYNTHESIS AND PHARMACOKINETIC ACTIVITIES OF PULMODIL AND PULMODIL-1, TWO CHLOROPHENYLPIPERAZINE SALT DERIVATIVES

(75) Inventor: Ing-Jun Chen, Kaohsiung (TW)

Correspondence Address: VOLPE AND KOENIG, P.C., UNITED PLAZA, 30 SOUTH 17TH STREET, PHILADELPHIA, PA, 19103, US

(73) Assignee: KAOHSIUNG MEDICAL UNIVERSITY, Kaohsiung City (TW)

(21) Appl. No.: 12/620,485

(22) Filed: Nov. 17, 2009

(30) Foreign Application Priority Data
Apr. 30, 2009 (TW) 098114469

(51) Int. Cl. A61K 31/496; C07D 473/28; C12Q 1/02

(52) U.S. Cl. 514/252.16; 544/276; 435/29

(57) ABSTRACT

A compound including a salt derivative of the chlorophenylpiperazine moiety is provided, wherein the chlorophenylpiperazine moiety is derived from the reaction of a xanthine and a piperazine. The salt derivative thereof is non-toxic to the tracheal smooth muscle cells (TSMCs) and can be intravenously-, orally- or sublingually-dosed into the mammals.

### FIELD OF THE INVENTION

The present invention relates to a method for preparing a chlorophenylpiperazine salt derivative, and particularly relates to a 7-[2-[4-(2-chlorobenzene)piperazinyl]ethyl]-1,3-dimethylxanthine.HCl (abbreviated as Pulmodil) obtained by a recrystallization method. Pulmodul can be applied in pharmacokinetic activities.

### BACKGROUND OF THE INVENTION

Xanthine-based compound can result in the opening of potassium channel of the cultured cells and aortic/vascular (Wu et al., 2001), corporeal carvenosa (Lin et al., 2002) and tracheal smooth muscle (Wu et al., 2004) relaxation of mammals. This xanthine-based compound also can inhibit the growth of human normal prostate cell line PZ-HPV-7 to arrest the cell cycle in G₀/G₁ phase and increase p21 expression (Liu et al., 2007). In addition, xanthine-based compound also can result in turn-off of voltage-dependent calcium channel and relaxation of cerebral blood vessels (Wu et al., 2005). Therefore, xanthine-based compound might have great benefit on treating high-pressured patients.

Therefore, xanthine-based compound has relatively great contribution on basic medicine and pharmacology. However, it takes considerations of pharmacokinetic parameters, such as salts, dosages, dosage forms, solvent polarity, interaction with other compounds and toxicity to organisms, and so on, when xanthine-based compound is developed as the clinical medicine. These parameters are not disclosed in the previous researches and cannot be supposed to from the published literatures by the skilled person. Therefore, the present invention is provided for overcoming the defect of clinical medicine in the prior art, obtaining a new pharmaceutical compound to be more acceptable by human or mammals and obtaining particular curing effect.

It is therefore attempted by the applicant to deal with the above situation encountered in the prior art.

### SUMMARY OF THE INVENTION

First of all, a chlorophenylpiperazine salt derivative, i.e. Pulmodil, which is obtained by reacting 2-chloroethyl theophylline with 2-chlorophenyl piperazine and then recrystallizing the intermediate therefrom, is provided in the present invention. Pulmodil can be used in pharmacokinetic research to screen the adequate dosages and dosage forms and is non-toxic to cells to be the clinical medicine.

In accordance with the first aspect of the present invention, a chemical compound comprising a formula I,

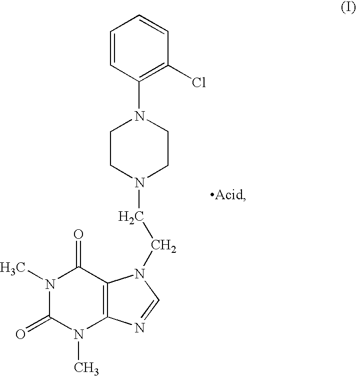

is provided.

Preferably, the acid is one of an organic acid and an inorganic acid.

Preferably, the organic acid can be citric acid, maleinic acid, fumaric acid, tartaric acid, oleic acid, stearic acid, benzenesulphonic acid, ethyl benzenesulphonic acid, benzoic acid, succinic acid, mesylic acid, dimesylic acid, acetic acid, propionic acid, pentanoic acid and aspartic acid.

Preferably, the compound is solved in anhydrous ethanol-polyethylene glycol-water when the acid is citric acid, and anhydrous ethanol-polyethylene glycol-water has a ratio of anhydrous ethanol:polyethylene glycol:water of 5:30:65, and the compound has a pH value ranged between 4.5 and 4.8.

Preferably, the inorganic acid is one selected from a group consisting of hydrochloride, sulfuric acid, phosphoric acid, boric acid and dihydrochloride.

Preferably, the compound is solved in a glucose solution when the acid is hydrochloride, the glucose solution has a weight/volume (w/v) concentration of 5%, and the compound has a pH value ranged between 5.8 and 6.4.

The chemical compound having formula I is a chlorophenylpiperazine salt derivative nominated as 7-[2-[4-(2-chlorobenzene)piperazinyl]-ethyl]-1,3-dimethylxanthine.acid. The acid is replaced as the above-mentioned organic acid or inorganic acid when the different organic or inorganic acid is used. For instance, the chlorophenylpiperazine salt derivative is nominated as 7-[2-[4-(2-chlorobenzene)piperazinyl]ethyl]-1,3-dimethyl-xanthine-hydrochloric acid and 7-[2-[4-(2-chlorobenzene)piperazinyl]-ethyl]-1,3-dimethylxanthine.citric acid respectively when the acid is referred to hydrochloric acid and citric acid.

Preferably, the compound further includes a pharmaceutically acceptable carrier.

In accordance with the second aspect of the present invention, a method for preparing a pharmaceutical compound having a formula I:

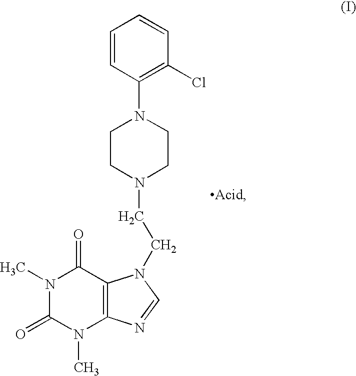

and the method comprising steps of: (a) boiling a theophylline and a piperazine to form a first mediator; (b) reacting the first mediator with an acid to form a first mixture; and (c) crystallizing the first mixture to obtain the pharmaceutical compound.

Preferably, the theophylline is a 2-chloroethyl theophylline and the piperazine is a 2-chlorophenyl piperazine.

Preferably, the step (a) is reacted in an ethanol solution having a first volume, the acid has a second volume, and the second volume is larger than the first volume.

Preferably, the ethanol solution is a hydrous ethanol solution.

Preferably, the step (a) further includes a step (a1) of reacting the mediator with a base to obtain an intermediate.

Preferably, the step (a) further includes a step (a2) of concentrating the intermediate as a powder.

Preferably, the base is sodium hydroxide or sodium hydrogen carbonate.

Preferably, the step (b) further includes a step (b1) of reacting the first mixture with an ethanol solution to form a saturated solution.

Preferably, the step (b) further includes a step (b2) of filtering the saturated solution to form a crystallized powder.

In accordance with the third aspect of the present invention, a method for evaluating a pharmacokinetic parameter of a compound, which is originated from the first aspect of the present invention, is provided. The method includes steps of: (a) providing a pharmaceutically effective amount of the compound to a subject; and (b) determining the pharmacokinetic parameter in a blood of the subject.

Preferably, the subject is a mammal being one selected from a group consisting of a human, a rat and a mouse, and the step (a) is performed by one selected from a group consisting of an oral administration, an intravenous injection and a sublingual administration.

The above objectives and advantages of the present invention will become more readily apparent to those ordinarily skilled in the art after reviewing the following detailed descriptions and accompanying drawings, in which:

### BRIEF DESCRIPTION OF THE DRAWINGS

FIGS. 1(A) and 1(B) respectively illustrate the comparison results between (A) 5 ng/ml Pulmodil and the standard and between (B) 200 ng/ml Pulmodil and the standard;

FIGS. 2(A) and 2(B) respectively depict the primary cultured tracheal smooth muscle cells (TSMCs) under (A) phase contrast microscope and (B) fluorescence microscope;

FIGS. 3(A) to 3(C) respectively illustrate the survival of different concentrations of Pulmodil, Pulmodil-1 and the major structural compound (MSC) on TSMCs of Wistar rat;

FIG. 4 illustrates a diagram showing the relationship between mean plasma concentration and time (hour), wherein male SD rats are given an intravenous (i.v.) injection (1.83 mg/kg), an oral administration (18.3 mg/kg) or a sublingual administration (3.4 mg/kg) of a single-dosed Pulmodil;

FIG. 5 illustrates a diagram showing the relationship between mean plasma concentration and time (hour), wherein male CD-1 (Crl) mice are given an i.v. injection (2.0 mg/kg) or oral administration (20.0 mg/ml) of a single-dosed Pulmodil; and

FIG. 6 illustrates a diagram showing the relationship between mean plasma concentration and time (hour), wherein male ICR mice are given an i.v. injection (2.0 mg/kg) or oral administration (20.0 mg/kg) of a single-dosed MSC.

### DETAILED DESCRIPTION OF THE PREFERRED EMBODIMENT

The present invention will now be described more specifically with reference to the following Embodiments. It is to be noted that the following descriptions of preferred Embodiments of this invention are presented herein for purpose of illustration and description only; it is not intended to be exhaustive or to be limited to the precise form disclosed.

The new chlorophenylpiperazine salt derivatives, Pulmodil and Pulmodil-1, are provided in the present invention, and their synthetic methods, physiochemical properties and pharmacokinetic activities are illustrated as follows.

### Embodiment 1

### Synthesis of Pulmodil

The first preferred embodiment of the present invention is Pulmodul. Method 1: 2-Chloroethyl theophylline, 2-chlorophenyl piperazine and sodium hydroxide (NaOH) (or sodium hydrogen carbonate (NaHCO₃)) were dissolved in hydrous ethanol solution based on the molecular weight percentage and heated under reflux for three hours. After overnight cooling, the supernatant was decanted for proceeding the vacuum concentration and dry process, and then one-fold volume of ethanol and three-fold volume of 2N hydrochloric acid (HCl) were added therein to dissolve at 50° C. to 60° C. as a saturated solution with pH 1.2. The saturated solution was sequentially decolorized with activated charcoal, filtered, deposited overnight and filtered to obtain Pulmodil with a white crystal. Pulmodil had a chemical formula as 7-[2-[4-(2-chlorobenzene)piperazinyl]ethyl]-1,3-dimethylxanthine.HCl, which had a melting point of 249° C. to 252° C. The reaction formula was illustrated as follows. Further, the purity of Pulmodil was determined with high performance liquid chromatography (HPLC).

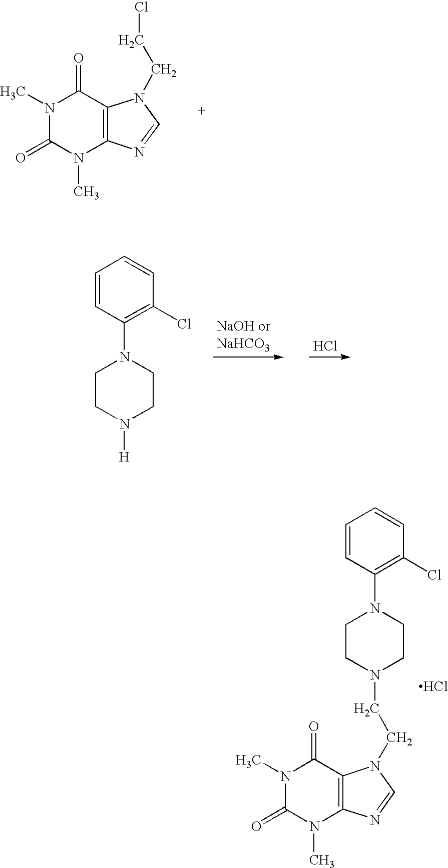

Method 2: 2-Chloroethyl theophylline and 2-chlorophenyl piperazine were dissolved in hydrous ethanol solution based on the molecular weight percentage and heated under reflux for three hours. After overnight cooling, the supernatant was decanted for proceeding the vacuum concentration and dry process, and then one-fold volume of ethanol and three-fold volume of 2N HCl were added therein to dissolve at 50° C. to 60° C. as a saturated solution with pH 1.2. The saturated solution was sequentially decolorized with activated charcoal, filtered, deposited overnight and filtered to obtain Pulmodil with a white crystal. Pulmodil had a chemical formula as 7-[2-[4-(2-chlorobenzene)piperazinyl]ethyl]-1,3-dimethylxanthine.HCl, which had a melting point of 249° C. to 252° C. The reaction formula was illustrated as follows.

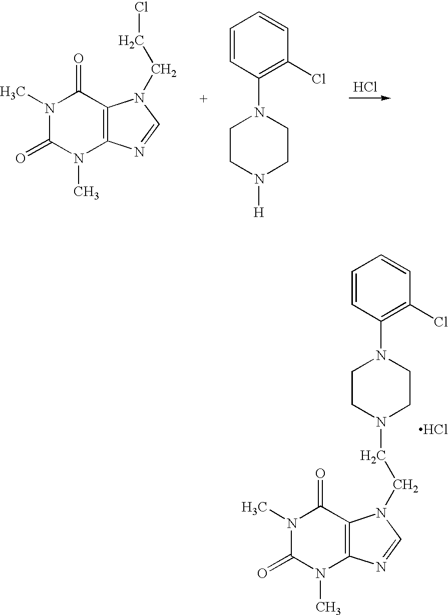

Regarding chemical structure, Pulmodil has a main structure and an HCl molecule. Although the main structure of Pulmodil could be obtained by reacting 7-ethylbromotheophylline with 1-(2-chlorophenyl)piperazine, Pulmodil could be obtained by the synthetic method disclosed in the present invention rather than the preparing steps, such as filtration and recrystallization, etc., of the major structured compound (hereinafter as “MSC”). “MSC” or “chlorophenylpiperazine main structure” illustrated in the present invention is referred to a compound that Pulmodil lacks an HCl molecule. The physiochemical and physiological differences between Pulmodil and its MSC were described in detail as follows.

(1) Melting Point:

The melting point of MSC was ranged between 168° C. and 172° C. which was significantly lower than that of Pulmodil (249° C. to 252° C.).

(2) HPLC Analysis:

In order to prove that Pulmodil functions as a single molecule in the organism rather than dissociates as two parts (i.e. MSC and HCl) to perform the respective functions, purity of Pulmodil solution was determined with HPLC. First, the Pulmodil and standard solutions with different concentrations were prepared, wherein the standard was a compound having a chemical formula, C₁₅H₁₄ClNO₂S.HCl and a molecular weight of 344.26. Pulmodil was dissolved in a solution containing 25% acetonitrile (CH₃CN) and 0.1% formic acid. After a serial dilution, a series concentrations (0, 0.1, 0.2, 0.5, 1, 2, 5, 10 and 20 ng/ml) of the standard solutions were used in HPLC to calculate the low-concentration standard curve, and a series concentrations (0, 10, 20, 50, 100, 200, 500, 1000, 2000 and 5000 ng/ml) thereof were used in HPLC to calculate the high-concentration standard curve. The standards were dissolved in a solution containing 50% acetonitrile, and then diluted with 100% acetonitrile. The concentration of the diluted Pulmodil was 18 ng/ml in the low-concentration standard curve and 450 ng/ml in the high-concentration standard curve, respectively. The column for HPLC was Luna C18 column (2.0 mm×50 mm, 5 mm, Phenomenex), in which the mobile phase included 23% acetonitrile and 1.0% formic acid, and the flow rate was about 0.2 ml/min.

The parameters of tandem mass spectrometry were set as capillary of 3.2 kV, Cone of 40 V, source temperature of 80° C., desolvation temperature of 400° C., collision of 20 V and multiplier of 500 V.

Pulmodil represented parent ion of 403.12 m/z (mass-to-charge ratio) and daughter ion of 222.95 m/z by the analysis of the tandem mass spectrometry, and standard represented parent ion of 308 m/z and daughter ion of 197.96 m/z thereby.

Please refer to Tables 1 and 2, which respectively depict the analytic results of the Pulmodil solutions with low- and high-concentrations in HPLC. The result of peak-covered area was calculated according to the retention time of the sample, and the area percentage represented the purity of the material in the sample. It could be known from Tables 1 and 2 that Pulmodil had 100% purity in the samples. It shows that Pulmodil is still a single compound after being dissolved.

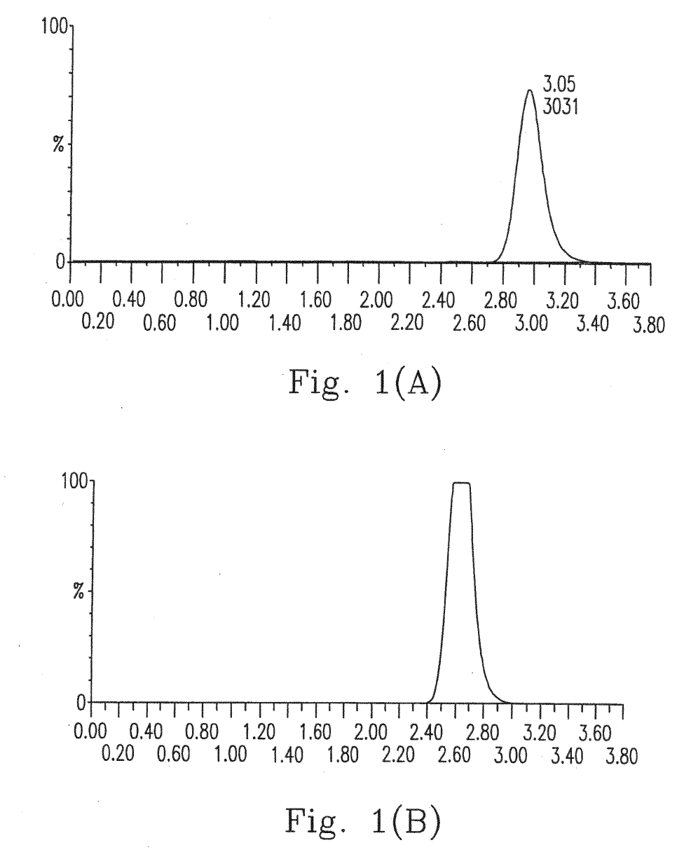

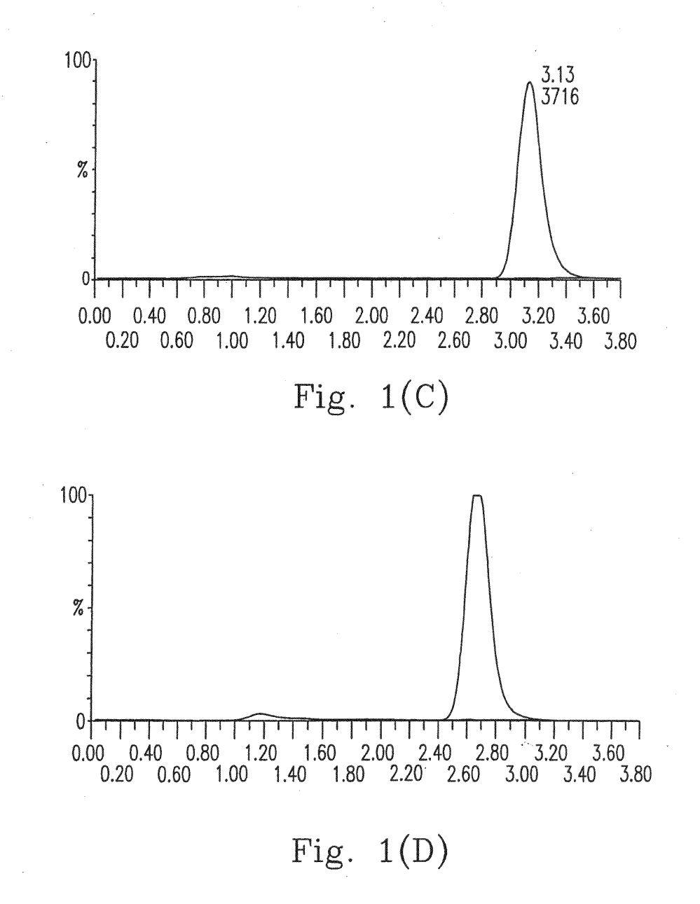

Please refer to FIGS. 1(A) and 1(B), which respectively illustrate the comparison results between (A) 5 ng/ml Pulmodil and the standard and between (B) 200 ng/ml Pulmodil and the standard. In FIG. 1(A), Pulmodil represented a peak at about 3 minutes, and no other minor peak was shown. It means that no other molecule exists in the solution.

TABLE 1
Result of low-concentration Pulmodil in HPLC

| | Retension | | | |
|---|---|---|---|---|
| Concentration (ng/ml) | time (min) | Area (uv × sec) | Area % | Height |
| 0.1 | 3.10 | 71.10 | 100 | 382 |
| 0.2 | 3.12 | 145.67 | 100 | 710 |
| 0.5 | 3.06 | 359.96 | 100 | 1759 |
| 1 | 3.09 | 801.44 | 100 | 3914 |
| 2 | 3.10 | 1463.25 | 100 | 7111 |
| 5 | 3.13 | 3716.39 | 100 | 17876 |
| 10 | 3.08 | 6919.70 | 100 | 34354 |
| 20 | 3.06 | 14481.06 | 100 | 71196 |

TABLE 2
Result of high-concentration Pulmodil in HPLC

| | Retension | | | |
|---|---|---|---|---|
| Concentration (ng/ml) | time (min) | Area (uv × sec) | Area % | Height |
| 10 | 3.08 | 143.95 | 100 | 706 |
| 20 | 3.04 | 320.91 | 100 | 1580 |
| 50 | 3.01 | 800.71 | 100 | 3955 |
| 100 | 3.01 | 1494.92 | 100 | 7303 |
| 200 | 3.05 | 3030.55 | 100 | 14663 |
| 500 | 3.05 | 7807.26 | 100 | 37646 |
| 1000 | 3.05 | 14499.72 | 100 | 70052 |
| 2000 | 3.01 | 28783.05 | 100 | 140845 |
| 5000 | 3.01 | 77175.41 | 100 | 368520 |

### Embodiment 2

### Synthesis of Pulmodil-1

The second preferred embodiment of the present invention is Pulmodul-1. Method 1: 2-Chloroethyl theophylline, 2-chlorophenyl piperazine and NaOH (or NaHCO₃) were dissolved in hydrous ethanol solution based on the molecular weight percentage and heated under reflux for three hours. After overnight cooling, the supernatant was decanted for proceeding the vacuum concentration and dry process, and then ethanol and citric acid at a ratio of 1:1 (mole/mole) were added therein to dissolve at 50° C. to 60° C. as a saturated solution with pH 4.0. The saturated solution was sequentially decolorized with activated charcoal, filtered and deposited overnight to obtain Pulmodil-1 with a white crystal. Pulmodil-1 had a chemical formula as 7-[2-[4-(2-chlorobenzene)-piperazinyl]ethyl]-1,3-dimethylxanthine.citric acid, and the reaction formula was illustrated as follows.

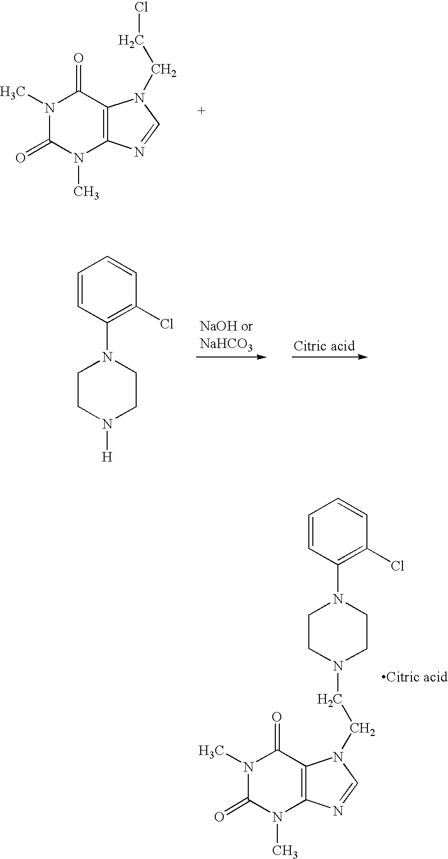

Method 2: 2-Chloroethyl theophylline, 2-chlorophenyl piperazine were dissolved in hydrous ethanol solution based on the molecular weight percentage and heated under reflux for three hours. After overnight cooling, the supernatant was decanted for proceeding the vacuum concentration and dry process, and then, ethanol and citric acid at a ratio of 1:1 (mole/mole) were added therein to dissolved at 50° C. to 60° C. as a saturated solution with pH 4.0. The saturated solution was sequentially decolorized with activated charcoal, filtered and deposited overnight to obtain Pulmodil-1 with a white crystal. Pulmodil-1 had a chemical formula as 7-[2-[4-(2-chlorobenzene)piperazinyl]ethyl]-1,3-dimethylxanthine.citric acid, and the reaction formula was illustrated as follows.

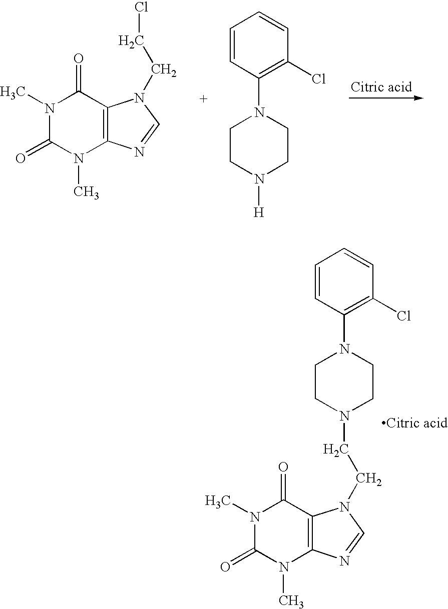

Pulmodil-1 was directly obtained by the synthetic method disclosed in the present invention rather than the preparing steps, such as filtration and recrystallization, etc., of MSC. The preparing process of MSC was significantly omitted in the technology of the present invention and the new manufacturing process was directly accomplished. Further, the reactant was 2-chlorophenyl piperazine, which would not result in the toxic gas production and draining risk, and the technology thereof was referred to the technology improvement.

Solubility Test:

Solubilities of three compounds, i.e. Pulmodil, Pulmodil-1 and MSC, disclosed in the present invention were evaluated and the results were shown in Table 3. It was found that Pulmodil-1 and MSC were necessary to be dissolved by using a surfactant. As shown in Table 3, Pulmodil-1 could be dissolved in the solution containing polyethylene glycol (PEG). However, Pulmodil-1 was dangerous for being the drug due to too much organic solvent contained in the solution. MSC could be dissolved in the solution containing propylene glycol. Pulmodil-1 and MSC solutions were acidic and were not adequate for the general physiological environment. Comparing with Pulmodil-1 and MSC, Pulmodil could be dissolved in 5% (w/v) glucose solution, had better solubility and was easily prepared. Pulmodil was safe in use since glucose solution was the popular medical supplement. Furthermore, the pH value of the dissolved Pulmodil was about 5.8 to 6.4, which closed to the general physiological environment.

TABLE 3
Solubilities of Pulmodil, Pulmodil-1 and MSC

| | Concentration | | |
|---|---|---|---|
| Compound | (mg/ml) | Solvent | pH |
| Pulmodil | 8 | 5% Glucose | 5.8-6.4 |
| Pulmodil-1 | 0.2 | Anhydrous EtOH:PEG | 4.5-4.8 |
| | | 400:H₂O = 5:30:65 (v/v) | |
| MSC | 100 | Anhydrous EtOH:PEG 400:1N | 4.2-4.8 |
| | | HCl:H₂O = 10:10:3:77 (v/v) | |
| MSC | 90 | 1.2 ml Propylene glycol | 4.2-4.8 |
| | | and 0.6 ml 1N HCl | |

In pharmacokinetics, bioavailabilities of Pulmodil and Pulmodil-1, respectively, were 0.2 and 0.04. Cytotoxicity from high to low was MSC, Pulmodil-1 and Pulmodil.

### EXPERIMENTAL METHOD

### I. Incubation of Rat Tracheal Smooth Muscle Cells (TSMCS)

1. Incubation of TSMCS

Tracheal tissue of Wistar rat (200 g to 250 g) was aseptically obtained and connective tissue around the tracheal tissue was removed. After clearance, tracheal tissue was aseptically sliced as fragments to spread on the T-25 flask and incubate in 6 ml DMEM medium (containing 20% (v/v) fetal bovine serum (FBS)) at 37° C. with 5% CO₂. Later, the medium was refreshed as medium B (DMEM supplemented with 10% FBS). The cells were sub-cultured when 80% to 90% cell confluence was achieved.

The medium was decanted at 80% to 90% cell confluence, and cells were rinsed with 2 ml phosphate buffered saline (PBS) once or twice. Next, 1 ml solution including 0.25% trypsin and 0.02% EDTA (ethylenediaminetetraacetic acid) was added to trypsinize cells at 37° C., and 10 ml medium B was added therein to cease the trypsin function. Supernatant was discarded after centrifugation, and cells were resuspended in 10 ml fresh medium B and cultured on 10 cm cull culture plate to proceed the subsequent experiments. The third to sixth generations of cells were used in the following experiments.

2. Identification of TSMCs:

First, morphology of smooth muscle cells was observed under an optical microscope, and then whether cells had a-actin was determined using immunofluorescent staining assay as follows.

The cover glass was aseptically disposed onto the well of 24-well culture plate and rat TSMCs of 5×10³ cells/ml were seeded into each well. The cells were incubated overnight at 37° C. with 5% CO₂ and 95% O₂ to make cells attach on the surface of the cover glass. The medium was withdrawn and cells were washed with ice-cold PBS for triplet, 0.5 ml for each time. Formaldehyde of 1 ml was added in each well to fix cells for 5 minutes at 37° C. PBS (1 ml) was added in each well after withdrawing formaldehyde, then the culture plate was gently shaken on horizontal orbital shaker for 5 minutes. The prepermeabilized lysis buffer (BD Pharmingen, San Diego, Calif.) of 0.5 ml was added in each well and reacted with cells for 15 minutes at room temperature after withdrawing PBS. The cells were washed with PBS for triplet, 0.5 ml for each time, after withdrawing the prepermeabilized lysis buffer. FITC-conjugated monoclonal mouse anti-smooth muscle a-actin antibody (0.5 ml, 1:100 dilution) was added into the culture plate to darkly react for 2 hours at room temperature. The wells were washed with washing buffer (containing 20 mM Tris base, 140 mM NaCl, 1% (v/v) Tween 20, pH 7.6) twice, and then the cover glass were mounted on the glass slide with fluorescent mounting medium for 30 minutes and cells were visualized using a fluorescent microscope.

3. Identification Result of Rat TSMCs:

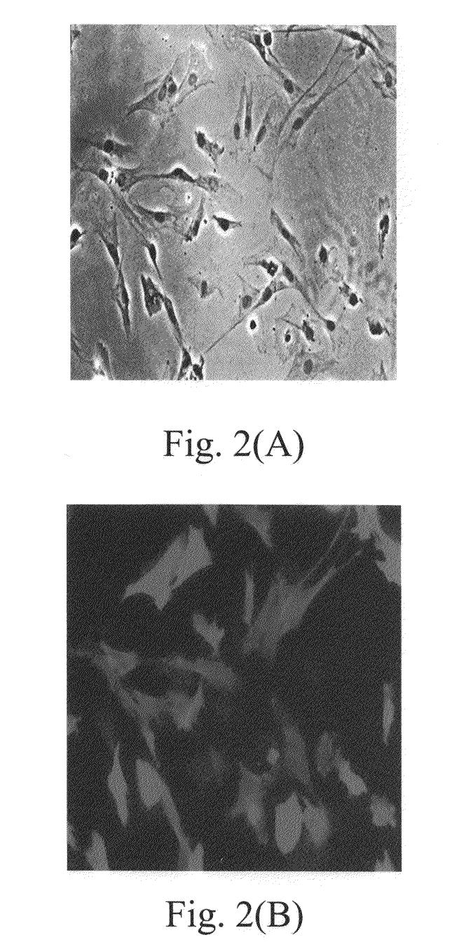

Please refer to FIGS. 2(A) and 2(B), which respectively depict the primary cultured TSMCs under (A) phase contrast microscope and (B) fluorescence microscope. Cells in FIG. 2(B) are identical with those in FIG. 2(A). It could be known from FIG. 2(B) that about 90% cells represented green fluorescence under fluorescence microscope, and it meant that the primary cells almost were smooth muscle cells and had a-actin.

### II. MTT Assay

Cell concentration was adjusted to 10⁴ cells/ml by using a cell counter. Ten thousand cells (1 ml) were inoculated in 24-well culture plate for 24 hours, and cells were attached thereon. Medium B then was added to culture cells for another 24 hours. Subsequently, different concentrations of drugs were added into wells for continuously incubating for 24 hours in low-oxygen incubator. MTT (methylhiazolyldiphenyl-tetrazolium bromide, 100 μl, 5 mg/ml) was added into the wells for darkly reacted for 3 hours at 37° C. Isopropanol of 500 μl was added to solve the crystal violet, formazan, after MTT was withdrawn. After 10 minutes, the supernatant (200 d) was transferred to the new 96-well culture plate to determine the absorbance at 540 nm (OD₅₄₀) and 630 nm (OD₆₃₀). The effect of drug on cellular growth was evaluated by the value of “OD₅₄₀-OD₆₃₀”.

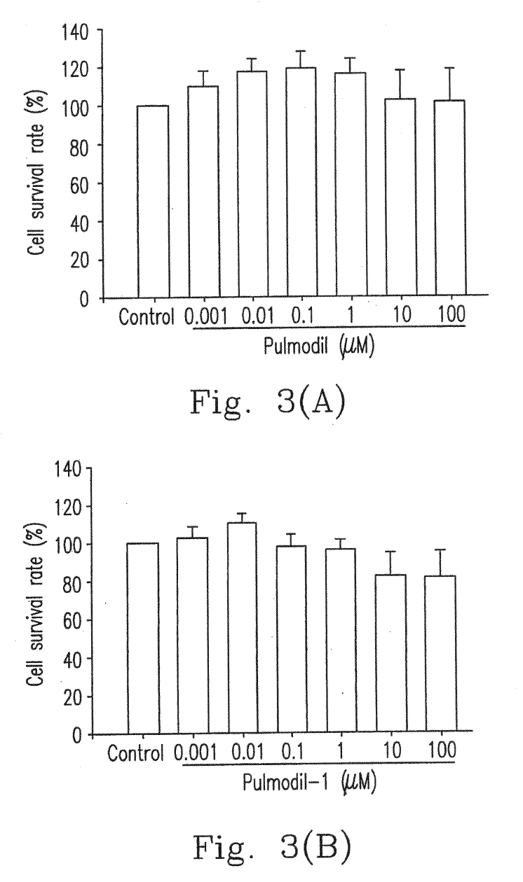

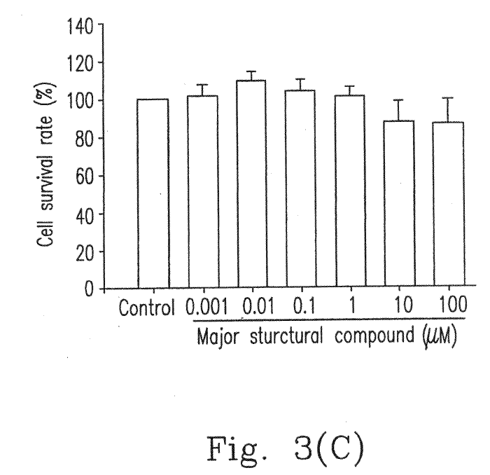

Please refer to FIGS. 3(A) to 3(C), which respectively illustrate the effects of different concentrations of Pulmodil, Pulmodil-1 and the major structural compound (MSC) on the survival rates of TSMCs of Wistar rat. In FIGS. 3(A) to 3(C), different concentrations (0.001 to 100 μM) of Pulmodil, Pulmodil-1 and MSC did not show cytotoxicity on rat TSMCs; in particular, the cell survival rate almost achieved 100% at high concentrations (10 and 100 μM). It meant that these three chlorophenyl piperazine salt derivatives were non-toxic to normal cells. Comparing with Pulmodil, Pulmodil-1 and MSC still represented few toxicity on cells, and cell survival rate was about 80% to 85% at high concentrations (10 and 100 μM).

Usage of Pulmodil was much safer than that of Pulmodul-1 or that of MSC in accordance with the results of cellular growth. Particularly, Pulmodil was the salt derivative of its major structural compound, and the structural difference between both compounds was a hydrochloride molecule. However, Pulmodil represented the significant improvement in toxicity and gained the great benefit on medical treatment.

### III. Pharmacokinetic Analysis

1. Preparation of Drugs:

To prepare Pulmodil, Pulmodil-1 and MSC for intravenous (i.v.) administration, Pulmodil (molecular weight (m.w.): 402.3) was dissolved in 5% glucose solution, Pulmodil-1 (m.w.: 595.1) was dissolved in ethanol-polyethylene glycol 400-water (a volume ratio of 5:30:65) solution, and MSC (m.w.: 402.9) was dissolved in 10% ethanol-10% propylene glycol-3% HCl mixture. The dosing concentrations of Pulmodil, Pulmodil-1 and MSC respectively were 0.37, 0.2 and 0.2 mg/ml. These pharmaceutical solutions were vortexed and sonicated to be fully dissolved to accomplish the preparation of drugs for i.v. administration. These solutions were stirred to the final dosing concentrations of Pulmodil, Pulmodil-1 and MSC of 1.83, 2.0 and 2.0 mg/ml, respectively.

In addition, Pulmodil was dissolved in 25 μl propylene glycol to prepare the sublingual dosing solution of 3.6 mg/kg.

2. Animal Experiment:

Male Sprague-Dawley (SD) rats (body weight: 250˜292 g) and male CD-1 (Crl) mice (body weight: 23˜25 g) were purchased from BioLasco Taiwan Co., Ltd., Taiwan, and male ICR mice (body weight: 23˜25 g) were provided from MDS Pharma Services (King of Prussia, Pa., U.S.A.). For i.v. administration, Pulmodil was administrated via tail vein with a bolus dosing volume of 1.25˜1.36 ml per SD rat (5 ml/kg), and Pulmodil-1 and MSC respectively were administrated via tail vein with a bolus dosing volume of 0.23 to 0.25 ml per CD-1 (Crl) mouse (10 ml/kg) and per ICR mouse (10 ml/kg). Rats and mice were fasted at least 16 hours prior to oral administration. For oral administration, Pulmodil was administrated via oral gavage with a dosing volume of 2.50˜2.76 ml per SD rat (10 ml/kg), and rats were allowed to standard chow for 4 hours post dosing. Pulmodil-1 and MSC respectively were administrated via oral gavage with a dosing volume of 0.23˜0.25 ml per CD-1 (Crl) mouse (10 ml/kg) and per ICR mouse (10 ml/kg), and mice were allowed to standard chow for 4 hours post dosing.

In addition, for sublingual administration, rat mouth was opened at the non-analgesia condition to allow application of Pulmodil (3.6 mg/kg/25 μl propylene glycol) dropped on sublingual cavity with micropipette within 1 minute.

3. Blood Sample Collection:

Regarding SD rats, blood samples were collected from the cannulated common carotid artery (CCA) post the i.v. administration for 0, 2, 5, 15, 30 minutes, 1, 1.5, 2, 4, 6, 9, 24 and 27 hours (a total of 13 sampling time points). Before oral administration, blood samples of SD rat were collected by cannulation. After oral administration and sublingual administration respectively for 2, 5, 10, 15, 30 minutes, 1, 1.5, 2, 4, 6, 9, 24 and 27 hours (a total of 14 sampling time points including pre-oral administration), blood samples of SD rat were collected by cannulation. Blood samples of 6 SD rats were collected at each time point from the cannulated CCA and collected in a pre-chilling 1.5-ml Eppendorf microcentrifuge tube containing anti-coagulant EDTA-K3, followed by centrifugation at 4° C., 1,500 g for 15 minutes. The plasma fraction was transferred to a clean microcentrifuge tube and stored at −70° C.

Regarding CD-1 (Crl) mice and ICR mice, blood samples were collected by cardiac puncture post i.v. administration for 0, 2, 5, 15, 30 minutes, 1, 1.5, 2, 4, 6, 9, 24 and 27 hours (a total of 13 sampling time points). Blood samples of mice were collected by cardiac puncture before oral administration. Blood samples of three mice were collected at each time point by cardiac puncture post oral administration for 15, 30 minutes, 1, 1.5, 2, 4, 6, 9, 24 and 27 hours (a total of 11 sampling time points). Blood sample (0.5˜0.7 ml) collected by cardiac puncture was placed in a 1.5-ml Eppendorf microcentrifuge tube containing 5 μl of anti-coagulant heparin sodium (5,000 I.U./ml), followed by centrifugation at 12,000 rpm at 4° C. for 10 minutes. The plasma fraction was transferred to a clean microcentrifuge tube and stored at −70° C.

4. Pharmacokinetic Calculation and Analysis:

Pulmodil, Pulmodil-1 and MSC respectively were metabolized as MSC, and the mean plasma concentration of MSC was determined by liquid chromatography-mass spectrometry/mass spectrometry (LC-MS/MS, Waters, Alliance 2790 LC and Macromass Quattro Ultima) method with a reversed-phase Luna C18 column or a reversed-phase Biosil® ODS column. Plasma sample was mixed with acetonitrile and centrifuged, and the supernatant was injected onto an LC column. The pharmacokinetic parameters were calculated from mean plasma concentration by WinNonlin Standard program (version 3.1, Pharsight Corp.).

5. Experimental Results of Pharmacokinetics:

Mean plasma concentration of MSC in male SD rats achieved to a highest peak (1263.1±181.4 ng/ml) within 2 minutes post single-dosed Pulmodil (2.0 mg/kg, i.v.), then mean plasma concentration thereof was decreased with the increasing time. Mean plasma concentration thereof was decreased to 0.7±0.3 ng/ml post 6-hour administration. Mean plasma concentration thereof could not be determined post 9-hour administration.

Mean plasma concentration of MSC in male SD rats was determined as 879.7 ng/ml within 2 minutes with single oral administration (20 mg/kg) of Pulmodil, and mean plasma concentration thereof achieved to a highest peak (1294.1 ng/ml) at 5 minutes. Then, mean plasma concentration thereof was decreased with the increasing time. Mean plasma concentration thereof was decreased to 5.9 ng/ml post 9-hour administration and decreased to 0.2±0.1 ng/ml post 24- and 27-hour administrations.

Mean plasma concentration of MSC in male SD rats was determined as 22.5±4.1 ng/ml within 2 minutes with single sublingual administration (3.6 mg/kg/25 μA propylene glycol) of Pulmodil, and mean plasma concentration thereof achieved to a highest peak (25.9±5.5 ng/ml) at 10 minutes. Then, mean plasma concentration thereof was decreased with the increasing time. Mean plasma concentration thereof was decreased to 0.7 ng/ml post 9-hour administration and could not be determined post 24- and 27-hour administrations.

Therefore, Pulmodil could enter into blood circulation of SD rats via i.v. administration, oral administration or sublingual administration, and mean plasma concentration of MSC metabolized from Pulmodil was gradually absorbed or metabolized with the increasing time.

Mean plasma concentration of MSC in male CD-1 (Crl) mice achieved to a highest peak (1425.4±138.2 ng/ml) within 2 minutes with single-dosed Pulmodil-1 (2.0 mg/kg, i.v.). Then, mean plasma concentration thereof was decreased with the increasing time. Mean plasma concentration thereof was decreased to 2.7±2.2 ng/ml post 2-hour administration and could not be determined post 4-hour administration.

Mean plasma concentration of MSC in male CD-1 (Crl) mice achieved to a highest peak (683.2±187.5 ng/ml) within 2 minutes with single oral administration (20.0 mg/kg) of Pulmodil-1. Then, mean plasma concentration thereof was decreased with the increasing time. Mean plasma concentration thereof was decreased to 11.9 ng/ml post 4-hour administration and could not be determined post 6-hour administration. It was suggested that Pulmodil-1 was entirely or almost absorbed and/or metabolized to be less than the sensitivity limit of LC-MS/MS.

Therefore, Pulmodil-1 could enter into blood circulation of CD-1 (Crl) mice via i.v. administration or oral administration, and mean plasma concentration of MSC metabolized from Pulmodil-1 was gradually absorbed or metabolized with the increasing time.

Mean plasma concentration of MSC in male ICR mice achieved to a highest peak (447.76±50.30 ng/ml) within 2 minutes with single-dosed of MSC (2.0 mg/kg, i.v.). Then, mean plasma concentration thereof was decreased with the increasing time. Mean plasma concentration thereof was decreased to 0.39 ng/ml post 24-hour administration.

Mean plasma concentration of MSC in male ICR mice achieved to a highest peak (205.92±56.94 ng/ml) within 2 minutes with single-dosed MSC (20.0 mg/kg, i.v.). Then, mean plasma concentration thereof was decreased with the increasing time. Mean plasma concentration thereof was decreased to 3.74±1.62 ng/ml post 9-hour administration and could not be determined post 24-hour administration. It was suggested that MSC was entirely or almost absorbed and/or metabolized to be less than the sensitivity limit of LC-MS/MS.

Therefore, MSC could enter into blood circulation of ICR mice via i.v. administration or oral administration, and mean plasma concentration of MSC was gradually absorbed or metabolized with the increasing time.

The above-mentioned experimental results are lists in Table 4. In Table 4, the highest mean plasma concentration of MSC was achieved within 2˜5 minutes regardless of the dosage forms of these three different piperazine derivatives (i.e. Pulmodil, Pulmodil-1 and MSC) administrated to rats or mice. It meant That these drugs could be rapidly metabolized as MSC to be absorbed into the blood circulation. Comparing three dosage forms of Pulmodil administrated to SD rats, it was found that oral administration could result in Pulmodil to be metabolized as MSC, which resided in SD rats for a longest period (9 hours), and it was suggested that MSC could display the sufficient effect in rats. The results that different time-effects and mean plasma concentrations can be achieved by various dosage forms of Pulmodil to SD rat give a hint that the dispenser is able to prepare different dosage forms of Pulmodil depending on the targets, administrations and the predicted reaction time.

Regarding i.v. or oral administration to CD-1 (CH) mice, it was found that mean residence time (MRT, 4 hours) of oral administration was longer than that (2 hours) of i.v. administration, but the highest mean plasma concentration (1425.4 ng/ml) of MSC with i.v. administration was twice than that (683.2 ng/ml) of MSC with oral administration. Therefore, i.v. administration of Pulmodil-1 is adopted if the dispenser wants to achieve pharmacodynamic effect within a short period.

Although MRT of MSC with i.v. administration on ICR mice was longer than that of MSC with oral administration thereon, mean plasma concentration (4.82±1.98 ng/ml) of MSC with i.v. administration for 2 hours was significantly lower than that (94.26±40.36 ng/ml) of MSC with oral administration for 2 hours. It meant that MSC not only achieved to the highest mean plasma concentration in the short period by i.v. administration, but also rapidly metabolized. MSC also could achieve to the highest mean plasma concentration in the short period by oral administration, but metabolism of MSC was relatively slow. Similarly, the dispenser can take considerations of the targets, administrations and the predicted reaction time to prepare different dosage forms of MSC.

In consideration of i.v. administration and oral administration, both Pulmodil and Pulmodil-1 (comparing with MSC) could achieve to the highest mean plasma concentrations of MSC in 2 minutes. Therefore, the chlorophenylpiperazine derivatives, Pulmodil and Pulmodil-1, could be prepared as various dosage forms to human or rodents (rats and mice) than MSC.

TABLE 4
Comparisons of mean plasma concentration of MSC within Pulmodil,
Pulmodil-1 and MSC respectively intravenously administrated to SD
rats, oral administrated to CD-1 (Cr1) mice and sublingual
administrated to ICR mice

| | Mean plasma | | | | | | |
|---|---|---|---|---|---|---|---|
| | concentration of | Drug/Animal | | | | | |
| | MSC (ng/ml) | Pulmodil/SD | | Pulmodil-1/CD- | | | |
| Administration | and time | rats | | 1 (Crl) mice | | MSC/ICR mice | |
| i.v. | Maximum | 1263.1 | (2 min) | 1425.4 | (2 min) | 447.76 | (2 min) |
| | Minimum | 0.7 | (6 hr) | 2.7 | (2 hr) | 0.39 | (24 hr) |
| | Not determined | ND* | (9 hr) | ND | (4 hr) | ND | (27 hr) |
| Oral | Maximum | 1294.1 | (5 min) | 683.2 | (2 min) | 205.92 | (2 min) |
| | Minimum | 5.9 | (9 hr) | 11.9 | (4 hr) | 3.74 | (9 hr) |
| | Not determined | 0.2 | (24 hr) | ND | (6 hr) | ND | (24 hr) |

| | | | | | |
|---|---|---|---|---|---|
| Sublingual | Maximum | 25.9 | (10 min) | — | — |
| | Minimum | 0.7 | (9 hr) | — | — |
| | Not determined | ND | (24 hr) | — | — |

*ND: not determined

Further, pharmaceutical parameters were calculated by WinNonlin Standard Program, and the results were illustrated in Tables 5 to 7. Table 5 showed pharmacokinetic parameters where the single i.v. dosing (2.0 mg/kg) of Pulmodil, Pulmodil-1 and MSC respectively was administrated into male SD rats, CD-1 (Crl) mice and ICR mice. In Table 5, the highest mean plasma concentration (Cₘₐₓ) of MSC metabolized from Pulmodil was 1785.0 ng/ml, and it meant that the largest amount of Pulmodil-1 which was prepared from MSC containing citric acid entered into mice. However, the initiated mean plasma concentration (C₀) of MSC with i.v. administration of MSC achieved to 515.8 ng/ml. The area under curve (AUC), which was defined by integrating plasma concentration curve of MSC to time, of Pulmodil was higher than that of Pulmodil-1 or that of MSC, and it meant that the absorbance rate of Pulmodil on SD rats was highest, and the drug amount of Pulmodil entered into SD rats was higher than that of Pulmodil-1 and that of MSC thereinto. Accordingly, systemic clearance (Cl) of Pulmodil on SD rats was lower than that of Pulmodil and that of MSC thereon. However, mean residence time (MRT) of MSC in mice was higher than that of Pulmodil and that of Pulmodil-1 therein. It meant that metabolism rate and absorbance rate of Pulmodil and Pulmodil-1 were relatively fast. Therefore, terminal half-lives (t_{1/2}) of Pulmodil and Pulmodil-1 in plasma were shorter than that of MSC. Therefore, if achievement of the long-term drug effect is desired, time interval between two Pulmodil and/or Pulmodil-1 i.v. administrations could be shortened by multiple i.v. administrations and/or the shortened time interval, so that Pulmodil and/or Pulmodil-1 could be easily absorbed and maintained in the animals.

TABLE 5
Pharmacokinetic parameters of single i.v. dosing (2.0 mg/kg) of
Pulmodil, Pulmodil-1 and MSC respectively on male SD rats, male
CD-1 (Crl) mice and male ICR mice

| Pharmacokinetic | | Pulmodil-1/CD-1 | |
|---|---|---|---|
| parameters | Pulmodil/SD rats | (Crl) mice | MSC/ICR mice |
| C₍₀₎ (ng/ml) | ND | ND | 515.8 |
| Cₘₐₓ (ng/ml) | ND | 1785.0 | ND |
| AUC_{(0-inf)} | 608 | 371.3 | 236.6 |
| (ng × hr/ml) | | | |
| MRT (hr) | 0.6 | 0.3 | 2.0 |
| Cl (ml/min/kg) | 51.7 | 90.0 | 140.9 |
| Vₛₛ (l/kg) | 1.8 | 1.4 | 17.2 |
| V_{z} (l/kg) | 3.4 | 2.0 | 79.0 |
| t_{1/2} (hr) | 0.8 | 0.3* | 6.5** |

ND: not determined
*t_{1/2} determined between 0.5 and 2 hours
**t_{1/2} determined between 9 and 24 hours

Table 6 shows pharmacokinetic parameters of single oral administrations of Pulmodil (18.3 mg/kg), Pulmodil-1 (20.0 mg/kg) and MSC (20.0 mg/kg) respectively on male SD rats, male CD-1 (Crl) mice and male ICR mice. In Table 6, the highest Cₘₐₓ metabolized from Pulmodil was 1390 mg/ml which had a lowest corresponded time (Tₘₐₓ) of 0.1 hour. It meant that Pulmodil could achieve to the highest mean plasma concentration within the shortest time by oral administration. AUC for Pulmodil was also higher than that for Pulmodil-1 or MSC, and it meant that amount of Pulmodil entering into SD rats was higher than that of Pulmodil-1 or MSC entering into mice. However, half-life (t_{1/2}) of Pulmodil was higher than that of Pulmodil-1 and MSC, it meant that residence time of Pulmodil in animal was longer (3.3 hour). In addition, since oral drug would be absorbed by animal's digestive system and then entered into blood circulation, it could be known from the calculation equation “mean absorption time (MAT)=MRT_{PO}−MRT_{IV}” that Pulmodil was absorbed longer and showed more sufficient efficiency than Pulmodil-1 and MSC. Pulmodil had bioavailability of about 19% and could be the excellent oral drug, and Pulmodil-1 was the second one.

TABLE 6
Pharmacokinetic parameters of single oral administrations of
Pulmodil (18.3 mg/kg), Pulmodil-1 (20.0 mg/kg) and MSC
(20.0 mg/kg) respectively on male SD rats, male CD-1
(Crl) mice and male ICR mice

| Pharmacokinetic | | Pulmodil-1/CD-1 | |
|---|---|---|---|
| parameters | Pulmodil/SD rats | (Crl) mice | MSC/ICR mice |
| Cₘₐₓ (ng/ml) | 1390 | 683.0 | 205.9 |
| AUC_{(0-inf)} | 1142 | 379.3 | 462.5 |
| (ng × hr/ml) | | | |
| Tₘₐₓ (hr) | 0.1 | 0.3 | 0.25 |
| MRT (hr) | 1.7 | 1.0 | 2.8 |
| t_{1/2} (hr) | 3.3 | 1.0* | 1.2** |
| Bioavailability | 18.8 | 10.2 | 19.5 |
| (%) | | | |
| MAT*** (hr) | 1.1 | 0.7 | 0.8 |

*t_{1/2} determined between 0.5 to 4 hours
**t_{1/2} determined between 4 to 9 hours
***MAT = MRT_{PO} − MRT_{IV}

Please refer to Table 7, which illustrates pharmacokinetic parameters of single sublingual administration (3.4 mg/kg) of Pulmodil on male SD rats. In Table 7, Pulmodil with sublingual administration could achieve to the highest mean plasma concentration within the short time (Tₘₐₓ=0.1 hour). However, comparing with i.v. and oral administration, the absorbance amount of Pulmodil by sublingual administration was relatively low, and bioavailability of Pulmodil was low (about 4%). The terminal half-life (t_{1/2}) of Pulmodil by sublingual administration was 2.3 hours. Similarly, since sublingual drugs would be absorbed by digestive system and then entered into blood circulation, mean absorption time (MAT=MRT_{SL}−MRT_{IV}) was calculated as 1.8 hours. Higher dose of Pulmodil could be prepared, or multiple sublingual administrations could be performed in consideration of sublingual administration, so as to achieve the predicted effect.

TABLE 7
Pharmacokinetic parameters of single sublingual administration
(3.4 mg/kg) of Pulmodil on male SD rats

| Pharmacokinetic parameters | Pulmodil/SD Rats |
|---|---|
| Cₘₐₓ (ng/ml) | 27 |
| AUC_{(0-inf)} (ng × hr/ml) | 42 |
| Tₘₐₓ (hr) | 0.1 |
| MRT (hr) | 2.4 |
| t_{1/2} (hr) | 2.3 |
| Bioavailability (%) | 3.7 |
| MAT* (hr) | 1.8 |

*MAT = MRT_{SL} − MRT_{IV}

From pharmacokinetic analytic results in Tables 5 and 6, it showed that single i.v. administration or oral administration of MSC did not have significant substantive benefit on drug development since Cl value of MSC with i.v. administration was highest (Cl=140.9 ml/min/kg). It meant that metabolism rate of MSC was faster. MSC also showed high volume of distribution at steady-state (Vss), the lower oral absorbance rate (Tₘₐₓ=0.25 hour) and the lower mean plasma concentration (Cₘₐₓ=250.9 ng/ml).

Regarding i.v. administration of Pulmodil-1 on CD-1 (Crl) mice, MSC in plasma had short half-life (t_{1/2} from 0.5 to 2 hours=0.3 hour), short clearance rate (Cl=90 ml/min/kg), low mean residence time (MRT=0.3 hour) and low volume of distribution (Vss=1.4 l/kg). Pulmodil-1 had fast oral metabolism rate (Tₘₐₓ=0.3 hour), but bioavailability was 10%.

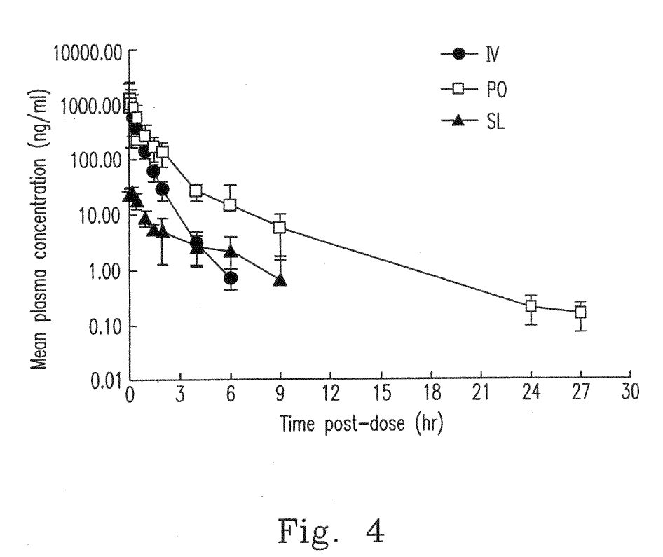

Please refer to FIG. 4, which illustrates a diagram showing the relationship between mean plasma concentration and time (hour), wherein the male SD rats are given an i.v. injection (1.83 mg/kg), a oral administration (18.3 mg/kg) or a sublingual administration (3.4 mg/kg) of a single-dosed Pulmodil. In FIG. 4, i.v. administration and oral administration could achieve relatively high mean plasma concentration of MSC after a short dosing time. In comparison, sublingual administration only achieved the relatively low mean plasma concentration of MSC (25.9 mg/ml). In addition, drug effect of oral administration on SD rats was higher than that of i.v. administration and sublingual administration. Therefore, Pulmodil was a relatively excellent choice to be orally administrated in animal.

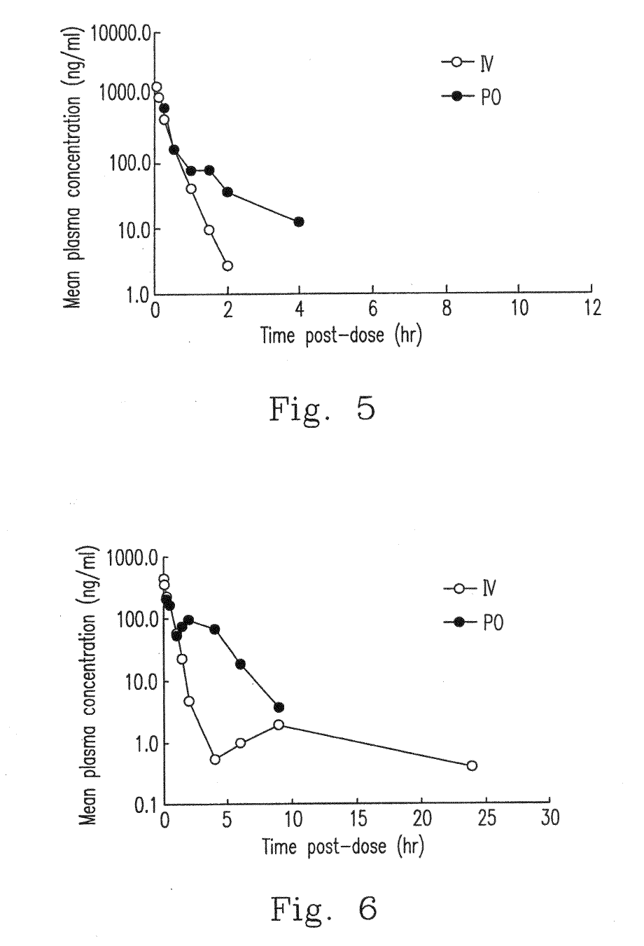

Please refer to FIG. 5, which illustrates a diagram showing the relationship between mean plasma concentration and time (hour), wherein the male CD-1 (Crl) mice are given an i.v. injection (2.0 mg/kg) or oral administration (20.0 mg/ml) of a single-dosed Pulmodil. In FIG. 5, the second absorbance peak was not shown in the group of intraveneously injected mice but was shown in the group of orally administrated mice at 1.5 hours. The metabolism rate from Pulmodil-1 to MSC in mice with i.v. administration was fast. The metabolism rate from Pulmodil-1 to MSC was slower and Pulmodil had long-term absorbance since oral-dosed Pulmodil-1 metabolized in the digestive system first and then entered into blood circulation.

Please refer to FIG. 6, which illustrates a diagram showing the relationship between mean plasma concentration and time (hour), wherein the male ICR mice are given an i.v. injection (2.0 mg/kg) or oral administration (20.0 mg/kg) of a single-dosed MSC. In FIG. 6, mean plasma concentration of MSC represented the second absorbance peak in the intravenously administrated (9 hours) and orally administration (2 hours) ICR mice. It meant that MSC in mice showed the phenomena of entero-hepatic reabsorption and multiple MSC absorbance.

In addition, mean plasma concentration of MSC that different dosage forms of Pulmodil were administrated to SD rats was also determined and analyzed. First, SD rats were divided into 3 groups, 8 rats per group. Next, 2.0 mg/kg Pulmodil, 3.6 mg/kg Pulmodil and 20 mg/kg Pulmodil respectively were intravenously administrated, sublingually administrated and orally administrated to SD rats, and blood samples were collected at different time points. Mean plasma concentration and pharmacokinetic analysis of MSC were determined by LC-MS/MS.

It was found that the highest mean plasma concentration of MSC in SD rats was achieved at 2˜5 minutes for i.v. administration (1263.12±181.44˜1171.077±222.16 ng/ml), at 2˜30 minutes for sublingual administration (22.48±4.09˜18.24±5.70 ng/ml) and at 5˜10 minutes for oral administration (954.32±332.81 to 806.85±160.05 ng/ml). Later, mean plasma concentration of MSC was decreased with the increasing time. Therefore, Pulmodil rapidly entered into SD rats via i.v. administration, effectively metabolized as MSC and was absorbed to enter into blood circulation.

In accordance with the above-mentioned experimental results, it was known that the best pharmacokinetic parameters of MSC was achieved in SD rats by orally administrating with Pulmodil, and thus Pulmodil could be the candidate for the actual medical trial. In addition, retention time of Pulmodil and Pulmodil-1 in mice with i.v. and oral administration respectively was different, thus retention time could be the indicator of dosage form and dosing time by the dispenser. Further, dispenser could take account for the various dosed persons to organize different ratio of Pulmodil and Pulmodil-1 to prepare as different dosage forms, and administrated to mammals such as human or rodents, so as to achieve the diagnostic effect.

Therefore, first, it is proved that the chlorophenylpiperazine salt derivatives prepared in the present invention cannot inhibit the growth of rat TSMCs, and further different dosed effects are obtained with i.v. administration or oral administration. Therefore, the dispenser is able to have multiple dosing choices to achieve anti-cancer effect in mammals, achieve the aortic, corporeal carvenosa and tracheal smooth muscle relaxation effects in mammals, or achieve the high pressure-treated effect. The above-mentioned chlorophenylpiperazine salt derivatives, Pulmodil and Pulmodil-1, can be the actual drugs than MSC to be dosed to animals.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

What is claimed is:

1. A chemical compound comprising a formula I:

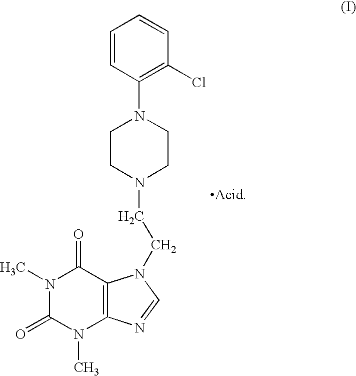

2. The compound according to claim 1, wherein the acid is one of an organic acid and an inorganic acid.

3. The compound according to claim 2, wherein the organic acid is one selected from a group consisting of a citric acid, a maleinic, acid, a fumaric acid, a tartaric acid, an oleic acid, a stearic acid, a benzenesulphonic acid, an ethyl benzenesulphonic acid, a benzoic acid, a succinic acid, a mesylic acid, a dimesylic acid, an acetic acid, a propionic acid, a pentanoic acid and an aspartic acid.

4. The compound according to claim 3, wherein the compound is solved in an anhydrous ethanol-polyethylene glycol-water when the acid is the citric acid.

5. The compound according to claim 4, wherein the anhydrous ethanol-polyethylene glycol-water has a ratio of anhydrous ethanol:polyethylene glycol:water of 5:30:65, and the compound has a pH value ranged between 4.5 and 4.8.

6. The compound according to claim 2, wherein the inorganic acid is one selected from a group consisting of a hydrochloride, a sulfuric acid, a phosphoric acid, a boric acid and a dihydrochloride.

7. The compound according to claim 6, wherein the compound is solved in a glucose solution when the acid is the hydrochloride.

8. The compound according to claim 7, wherein the glucose solution has a weight/volume concentration of 5%, and the compound has a pH value ranged between 5.8 and 6.4.

9. The compound according to claim 1 further comprising a pharmaceutically acceptable carrier.

10. A method for preparing a pharmaceutical compound having a formula I: 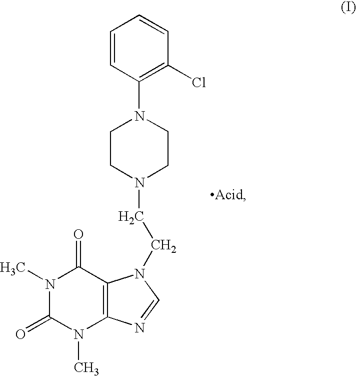
and the method comprising steps of
(a) boiling a theophylline and a piperazine to form a first mediator;
(b) reacting the first mediator with an acid to form a first mixture; and
(c) crystallizing the first mixture to obtain the pharmaceutical compound.

11. The method according to claim 10, wherein the theophylline is a 2-chloroethyl theophylline and the piperazine is a 2-chlorophenyl piperazine.

12. The method according to claim 10, wherein the step (a) is reacted in an ethanol solution having a first volume, the acid has a second volume, and the second volume is larger than the first volume.

13. The method according to claim 12, wherein the ethanol solution is a hydrous ethanol solution.

14. The method according to claim 10, wherein the step (a) further comprises a step (a1) of reacting the mediator with a base to obtain an intermediate.

15. The method according to claim 14, wherein the step (a) further comprises a step (a2) of concentrating the intermediate as a powder.

16. The method according to claim 14, wherein the base is one of a sodium hydroxide and a sodium hydrogen carbonate.

17. The method according to claim 10, wherein the step (b) further comprises a step (b1) of reacting the first mixture with an ethanol solution to form a saturated solution.

18. The method according to claim 17, wherein the step (b) further comprises a step (b2) of filtering the saturated solution to form a crystallized powder.

19. A method for evaluating a pharmacokinetic parameter of a compound as claimed in claim 1, comprising steps of:
(a) providing a pharmaceutically effective amount of the compound to a subject; and
(b) determining the pharmacokinetic parameter in a blood of the subject.

20. The method according to claim 19, wherein the subject is a mammal being one selected from a group consisting of a human, a rat and a mouse, and the step (a) is performed by one selected from a group consisting of an oral administration, an intravenous injection and a sublingual administration.
